(19) United States
(12) Patent Application Publication
(10) Pub. No.: US 2022/0054236 A1
(43) Pub. Date: Feb. 24, 2022

(54) COMPRESSION TOOL FOR DENTAL IMPLANTATION SITES, AND A METHOD OF USING THE SAME

(71) Applicant: CARMEX PRECISION TOOLS LTD., MAALOT INDUSTRIAL ZONE (IL)

(72) Inventors: OMER JACOBY, MAALOT INDUSTRIAL ZONE (IL); YUVAL COHEN, MAALOT INDUSTRIAL ZONE (IL)

(21) Appl. No.: 17/428,755

(22) PCT Filed: Feb. 5, 2019
(86) PCT No.: PCT/IB2019/000125

§ 371 Date: Aug. 5, 2021

(51) Int. Cl. A61C 8/00; A61C 3/02; A61B 17/16

(52) U.S. Cl. CPC A61C 8/0089; A61B 17/1615; A61C 3/02

(57) ABSTRACT

A compression tool for performing a finishing procedure, within a bore hole formed within a human jawbone so as to serve as an implantation site for a dental implant, by compression and densification processes, comprises a tapered body portion, and a plurality of flute sections disposed the body portion, wherein, when the compression tool is rotated in a clockwise direction and simultaneously axially inserted into the bore hole, the plurality of flute sections, which comprise structure for continuously cutting bone material from bone mass surrounding and defining the bore hole formed within the human jawbone, for accumulating the bone material so as to prevent the bone material from being evacuated from the implantation site, and for immediately compressing the bone material, cut from the bone mass surrounding and defining the bore hole formed within the human jawbone, back into the bone mass surrounding and defining the bore hole formed within the human jawbone will compress, compact, and enhance the density of the bone mass surrounding and defining the bore hole formed within the human jawbone.

### FIELD OF THE INVENTION

The present invention relates generally to tools, and more particularly to a new compression tool for use in connection with the enhancement of the thickness and density of the bone sections forming or surrounding a dental implantation site.

### BACKGROUND OF THE INVENTION

Various medical drills or osteotomes are of course known in the art. Examples of some known drills or osteotomes can be appreciated from U.S. Pat. No. 10,039,621 which issued to Huwais on Aug. 7, 2018; U.S. Pat. No. 9,028,253 which issued to Huwais on May 12, 2015; and U.S. Pat. No. 9,022,783 which issued to Huwais on May 5, 2015. Still yet further, other drills or osteotomes are disclosed within the Versah® manual entitled Densah® Bur Surgical Technique Manual. As noted within the Huwais U.S. Pat. No. 9,022,783, there is disclosed an apparatus and method for enlarging an existing osteotomy by utilizing a plurality of drills or osteotomes having progressively larger diametrical extents which will incrementally enlarge the original osteotomy until the desired diametrical extent of the osteotomy, for accommodating the dental implant, is achieved. In accordance with the disclosed method of forming an osteotomy as disclosed within the Huwais patent, each drill or osteotome comprises a plurality of flutes having burnishing edges located at the crest portions of the plurality of flutes. The burnishing edges are provided for achieving a burnishing process wherein the internal wall surfaces of the bone, surrounding and defining the osteotomy, are effectively plastically deformed as a result being simultaneously expanded and compressed with little or no removal of bone material. However, there are several drawbacks to the system and method disclosed within these patents.

For example, in addition to the plurality of flutes having the plurality of burnishing edges located upon the crest portions of the flutes, the drills or osteotomes of the Huwais patent also comprises cutting flutes such that when the drill or osteotome is rotated in the opposite direction, the osteotomy may be enlarged further by actually cutting or excavating bone material from the osteotomy as opposed to utilizing compression and plastic deformation techniques. In addition, it is noted that in accordance with the preparation of the dental implantation site, several drills or osteotomes, having progressively larger diametrical extents, are utilized such that the diametrical extent of the osteotomy is incrementally increased until the diametrical extent of the osteotomy has attained a predetermined size or diametrical extent so as to accommodate the dental implant. The use of such multiple drills or osteotomes renders the entire borehole drilling and finishing or burnishing process unnecessarily prolonged and tedious since the dental surgeon will need to periodically exchange several drills or osteotomes for other drills or osteotomes, having incrementally larger diametrical extents, until the drilled borehole has achieved the desired diametrical extent. Still further, it is noted that in accordance with the principles and teachings of this patent to Huwais, the drill or osteotome comprises a plurality of flutes which are equiangularly spaced from each other as considered in a circumferential array. This equiangularly spaced, circumferential array of the flutes has been discovered to result in the chattering or vibration of the drill or osteotome as it undergoes its rotational movement. Lastly, it is also noted from the disclosure of this patent that the surgeon will employ a “bouncing” technique wherein the surgeon will axially reciprocate the drill or osteotome as a result of which downwardly oriented axial pressure is applied to the drill or osteotome in short bursts with the surgeon continuously monitoring the progress of the drilling or burnishing process and making fine corrections and adjustments as he deems necessary. It is known in the art that such monitoring techniques are conventionally employed by dental surgeons so as to ensure that the drill or osteotome has not reached depth levels beyond the intended depth level, which could be dangerous. For example, in accordance with some known drills or osteotomes, depth gauge numerical markings are provided upon the fluted portion of the drill or osteotome in order to permit the dental surgeon to visually determine the depth to which he has drilled. However, not only are such markings difficult to see during the actual drilling process, but in addition, such entails an obvious discontinuous procedural process of drilling, determining the depth to which the dental surgeon has drilled, continue drilling deeper, again determining the depth to which the dental surgeon has drilled further, and the like, thereby rendering the drilling process imprecise, tedious, and prolonged.

The disclosure found within the Huwais U.S. Pat. No. 9,028,253 is essentially the same as that found within the Huwais U.S. Pat. No. 9,022,783 with the additional disclosure that the burnishing procedure is accomplished as a result of the rotation of the drill or osteotome in the counterclockwise direction, while the cutting procedure is accomplished by rotation of the drill or osteotome in the clockwise direction, and that the surgeon can quickly switch from burnishing to cutting or vice versa by switching the drive control upon the drive motor used to rotationally drive the drill or osteotome. Again, however, this start, stop, and switch procedure is somewhat tedious and prolongs the formation of the desired osteotomy. Lastly, the disclosure found within the Huwais U.S. Pat. No. 10,039,621 is likewise essentially the same as the disclosures of the previous two Huwais patents with the additional disclosure that the drill or osteotome can be concurrently utilized with high-frequency vibrational techniques whereby ultrasonic vibrations are utilized to pulverize portions of the bone. Still further, the drill or osteotome of this Huwais patent discloses a pair of circumferentially offset lips which terminate in a sharply pointed chisel point which can pose a significant danger during surgery as will be discussed more fully hereinafter.

Considering next the procedural manual of Versah®, as was also noted within the aforenoted patents to Huwais, it is known that when implanting a dental implant, in addition to drilling or cutting the hole into which the implant is to be implanted, it is critically important that the bone forming or surrounding the implantation site be compacted or densified in order to substantially improve the strength, integrity, and stability of the bone defining the implantation site within which the implant is to be implanted, as well as to improve the subsequent healing process of the implantation site. While the particular drills or osteotomes of Versah®, as disclosed within the aforenoted manual, are operatively satisfactory, significant improvements can be made with respect to the tool per se as well as the procedural usages thereof. For example, as was true with the drills or osteotomes of the various Huwais patents, the drill or osteotome of Versah® is likewise in fact a drill or osteotome wherein the drill or osteotome is utilized to form the borehole within the implantation site as a result of clockwise rotational operation of the drill or osteotome, while subsequently, the drill or osteotome must be rotated in the counterclockwise direction in order to achieve the compaction or densification of the bone structure immediately surrounding and defining the borehole of the implantation site.

Still yet further, in accordance with the aforenoted manual, the drill or osteotome of Versah® is rotated at relatively high speeds comprising 800-1500 RPM, and requires constant water irrigation in order to prevent the implantation site from being subjected to significantly elevated temperatures, or else, such significantly elevated temperatures could potentially lead to the development of gangrene or other problems at the implantation site. The problem with utilizing or needing continuous water irrigation, however, is that, in addition to being cumbersome while simultaneously drilling the borehole within the implantation site by means of the osteotome or drill, the use of irrigation water is in fact difficult to effectively achieve as a coolant within the lowermost depths of the drilled borehole where the vast majority of the heat is being generated as a result of the drilling process. This is because of the fact that a vast majority of the irrigation water will not actually enter the drilled borehole or implantation site but, to the contrary, will effectively be deflected away from the drilled borehole or implantation site by means of the drill or osteotome itself. This is an example or corollary of the basic Pauli Exclusion Principle which states that “Two objects cannot occupy the same space at the same time.”

An additional operational drawback of the Versah® drill or osteotome is that when using the drill or osteotome, the dental practitioner needs to stop the drilling at various times in order to determine how far the drill or osteotome has drilled the borehole within the jawbone. As has been noted hereinbefore, this type of operation can be somewhat tedious and time-consuming. One additional reason that the dental practitioner must follow this particular procedure when utilizing the drill or osteotome of Versah® resides in the fact that, similar to the drill or osteotome disclosed within the Huwais U.S. Pat. No. 10,039,621, the drill or osteotome of Versah® has a sharply-pointed or substantially conically configured distal end face which is obviously utilized to begin the drilling process at the implantation site. This structure, incorporated upon the distal end face of the drill or osteotome, can be potentially dangerous, however, in view of the fact that within the human skull, and more particularly within the human mandible or lower jawbone, or within the human maxilla or upper jawbone, various sinus cavities and/or membranes are present. Improper or prolonged usage of the drill or osteotome, such as that illustrated within the aforenoted manual or Versah®, can potentially permit the sharply-pointed or conically configured distal end face of the drill or osteotome to easily pierce or penetrate such membranes and enter the sinuses with unwanted or undesirable deleterious effects such as, for example, within the upper jawbone or maxilla, such improper or prolonged usage of the drill or osteotome, with piercing of the membrane or intrusion into one of the sinus cavities can cause deafness. In a similar manner, piercing of the membrane within the mandible or lower jaw can potentially lead to lockjaw.

A need therefore exists in the art for a new compression tool which has been specifically developed for use in connection with osteotomies formed within a human jawbone in connection with the formation of a dental implantation site. Another need exists in the art for a new compression tool which will overcome the structural and procedural deficiencies of known prior art tools that have been used in connection with the formation of dental implantation sites. Yet another need exists in the art for a new compression tool which is specifically structured so as to be used exclusively as a finishing tool in connection with the formation of dental implantation sites. Still yet another need exists in the art for a new compression tool which is specifically structured so as to be used exclusively as a finishing tool in connection with the formation of dental implantation sites, and wherein the tool is only required to be rotated in a single direction, that is, in the normal clockwise direction, in order to accomplish its finishing procedures which importantly include the compression, compaction, or densification of the bone material surrounding, or residually defining, a previously formed borehole within the dental implantation site.

A still further need exists in the art for a new compression tool which is specifically structured so as to be used exclusively as a finishing tool in connection with the formation of dental implantation sites, and wherein, more specifically, the new compression tool is structured such that the material removed from the innermost circumferential wall of the implantation site is not evacuated, but instead, is effectively entrapped and forced to move in a circumferential manner such that the compression tool can then cause such bone material to be subsequently compressed into the surrounding bone material that effectively defines the implantation site so as, to compress, compact, and further densify the surrounding bone material that effectively defines the implantation site. A yet still further need exists in the art for a new compression tool which is specifically structured so as to be used exclusively as a finishing tool in connection with the formation of dental implantation sites, and wherein the tool is able to be operated at relatively low or moderate rotational speeds so as not to generate excessive heat which, in turn, will not require the usage of a substantial amount of water irrigation.

Still yet further, a need exists in the art for a new compression tool which is specifically structured so as to be used exclusively as a finishing tool in connection with the formation of dental implantation sites, and wherein the tool is able to be operated continuously so as not to unnecessarily prolong the borehole finishing process. Another need exists in the art for a new compression tool which is specifically structured so as to be used exclusively as a finishing tool in connection with the formation of dental implantation sites, and wherein the tool is operated continuously so as not to unnecessarily prolong the surgical procedure, and without any reciprocal, upward and downward, or back and forth “bouncing” movements, such that the finishing, compression, compaction, or densification process is completed in a single pass of the tool within and through the dental implantation site. Still another need exists in the art for a new compression tool which is specifically structured so as to be used exclusively as a finishing tool in connection with the formation of dental implantation sites, and wherein there is no fear of the distal end face of the drill or osteotome piercing any of the membranes present within either one of the human jawbones and thereby entering any of the sinus regions defined within the human skull.

An additional need exists in the art for a new compression tool which is specifically structured so as to be used exclusively as a finishing tool in connection with the formation of dental implantation sites within human jawbones, and wherein the perfectly desired size or diametrical extent of the borehole, defining the implantation site, is achieved by means of utilizing only a single compression tool, in lieu of utilizing a plurality of tools having progressively larger diametrical extents, so as to permit the finishing process to be readily accomplished with a minimal amount of ease, since multiple tools need not be exchanged, and within a minimal amount of surgical time. In a similar manner, a need exists in the art for a new compression tool which is specifically structured so as to be used exclusively as a finishing tool in connection with the formation of dental implantation sites within human jawbones, and wherein the tool, having the perfectly desired selectable length, is able to be selectively used, based upon x-ray or panoramic views of the patient's jawbone within which the implantation site is to be formed, so as to permit the finishing process to be readily accomplished without any fear of piercing any membranes or entering any sinus cavities defined within the jawbone or skull of the patient. Lastly, a need exists in the art for a new compression tool which is specifically structured so as to be used exclusively as a finishing tool in connection with the formation of dental implantation sites within the human jaw, and wherein the flutes, defined upon the lower or distal end shank portion of the compression tool, are irregularly spaced as considered in the circumferential direction so as to eliminate any chatter or vibration during the surgical procedure.

### SUMMARY OF THE INVENTION

The foregoing and other needs are satisfied by means of the new compression tool which comprises a shank or body portion defined around a longitudinally extending axis, and wherein the lower section of the shank or body portion comprises a plurality of longitudinally extending left-handed flute structures disposed around the external surface portion of the lower section of the shank or body portion within a circumferential array but wherein the plurality of flute structures are not spaced equiangularly around the longitudinal axis of the drill or osteotome. More particularly, it is seen that the plurality of flute structures may, for example, comprise six (6) flute structures, although the precise number of flute structures may vary, and that each flute structure comprises a right-hand leading crest or cutting edge portion, and trailing margin and relieved land portions. The margin portion is effectively disposed within the same tangential plane or circumferential locus as that of the leading crest or cutting edge portion, as considered in connection with the inner circumferential wall of the bone material defining the dental implantation site, while the relieved land portion is angled away from the tangential leading crest or cutting edge portion, and the margin portion, so as to effectively reduce the surface area disposed in contact with the inner circumferential wall of the bone material defining the dental implantation site which thereby, in turn, reduces the amount of heat generated during the compression or densification process of the procedure. The leading crest or cutting edge portion also leads into an arcuately configured groove or pocket section of each flute structure so as to, effectively entrap and accumulate minute particles of bone which have been removed by means of the leading crest or cutting edge portions of the flute structures.

As a result of this integral composite flute structure, comprising the left-handed flutes with the right-handed cutting edges, as well as the margin, relieved, and grooved or pocket sections, the leading crest or cutting edge portions will therefore effectively remove minute particles of bone from the innermost circumferential wall surface of the bone surrounding the dental implantation site, such particles will then be effectively prevented from being evacuated from the implantation site so as to accumulate within the arcuately configured groove or pocket sections of the flute structures, and the margin portions will then compress such minute particles of bone radially outwardly and into the bone material circumferentially surrounding the implantation site, thereby enhancing the densification of such bone material circumferentially surrounding and defining the implantation site by means of compression and compaction. It is additionally noted that the lower fluted section of the shank or body portion is tapered a predetermined amount as one proceeds from the distal end, at which the flute structures begin, to the apical end, which is adapted to be inserted within a rotary motor or other power source so as to impart rotational movement to the compression tool, it being appreciated that the distal end of the compression tool is provided with the smallest diametrical extent. It is further noted, that in conjunction with such tapered fluted section, and the dimensions of the diametrical extents of the flute structures at any axial location along the longitudinally axial extent of the compression tool, that one side of the upper, non-fluted section of the shank or body portion has the diametrical extents, from smallest to largest, laser-marked upon an external surface portion of such upper, non-fluted section of the shank or body portion of the compression tool, while the diametrically opposite side of the upper, non-fluted section of the shank or body portion has the length of the fluted portion of the compression tool laser-marked upon its external surface portion. In this manner, the surgeon will know precisely which compression tool he will need or want to select in order to perform a particular finishing procedure in connection with the implantation site, both in terms of the length of the finished borehole to be formed within the implantation site, as well as the finished diametrical extent of the borehole formed within the implantation site.

In conjunction with such laser markings of the diametrical and length dimensions of the compression tool, the compression tool is also integrally provided with a circumferentially extending flanged portion at an axial location which separates the lower fluted section of the compression tool from the upper non-fluted section of the compression tool. This circumferentially extending flanged portion serves two purposes—the first purpose is that it effectively serves as a “physical barrier” or “stopper” whereby the dental practitioner knows that for the particular implantation site that he is operating on, further axial penetration of the compression tool into the implantation site is not to be pursued or else injury to the implantation site, or membranes or sinus regions within the jawbone or skull, may occur. The second purpose of the circumferentially extending flanged portion of the compression tool is that, in conjunction with the aforenoted laser markings indicating the taper of the compression tool, and the minimum and maximum diametrical extents of the lower fluted section of the compression tool, the circumferentially extending flanged portion is provided with two color-coded bands thereby further providing the dental practitioner with an additional visual indication of the fact that the compression tool is in fact tapered, and what the minimum and maximum diametrical extents of the compression tool are as are also noted upon the non-fluted shank portion of the coompression tool. Lastly, it is also noted that, in conjunction with the “stopper” function of the circumferentially extending flanged portion of the compression tool, the distal end face of the compression tool is substantially flat so as to effectively ensure that the fluted end of the drill or osteotome cannot in fact pierce any membranes or enter any sinus cavities.

### BRIEF DESCRIPTION OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1a is a first perspective view of a new compression tool as constructed in accordance with the principles and teachings of the present invention, and as viewed from a first side of the compression tool;

FIG. 1b is a second perspective view of the new compression tool as constructed in accordance with the principles and teachings of the present invention, and as viewed from a second opposite side of the compression tool;

FIG. 2 is a side elevational view of the new compression tool as illustrated within FIG. 1a;

FIG. 3 is a cross-sectional view of the new compression tool as illustrated within FIG. 2 and as taken along the line 3-3 of FIG. 2; and

FIG. 4 is an enlarged view of one of the flute sections as disclosed within the encircled region 4 of FIG. 3 showing, in greater detail, the flute, the cutting edge, the margin portion, and the relieved land portion of the overall flute structure.

### DETAILED DESCRIPTION OF THE PREFERRED EMBODIMENT

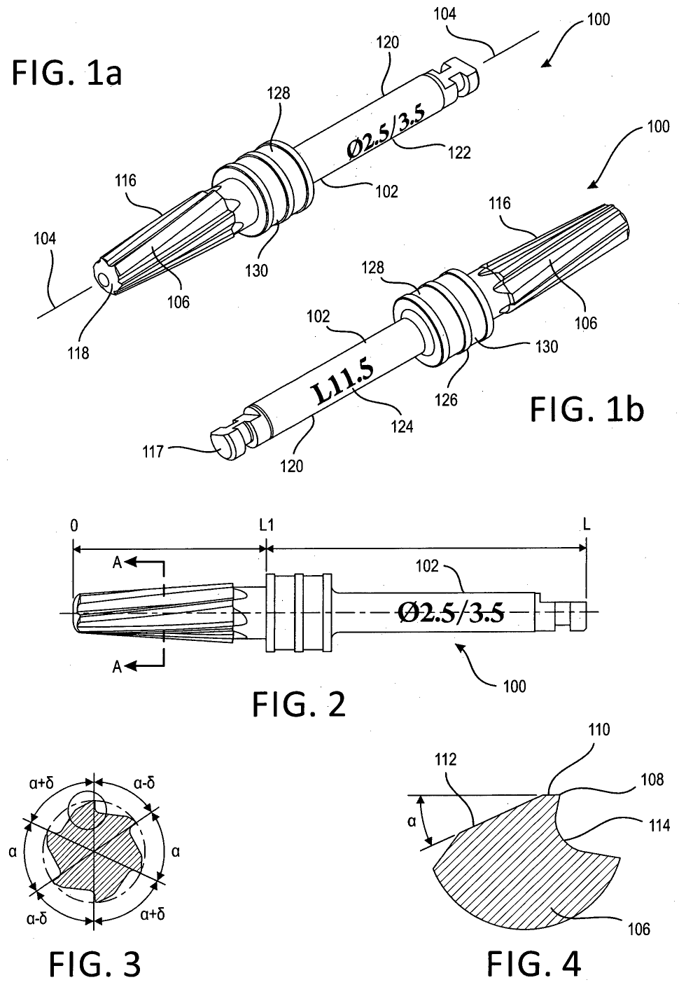

With reference now being made to the drawings, and more particularly to FIGS. 1a-4, a new compression tool is disclosed and is generally indicated by the reference character 100. More particularly, it is seen that the new compression tool 100 comprises a shank or body portion 102 which is defined around a longitudinally extending axis 104, and wherein the lower section of the shank or body portion 102 comprises a plurality of longitudinally extending flute sections 106 disposed around the external surface portion of the lower section of the shank or body portion 102 within a circumferential array. It is to be noted that the plurality of flute sections 106 are not spaced equiangularly around the longitudinal axis 104 of the drill or osteotome 100 as can best be appreciated from FIG. 3, wherein it is clearly illustrated that the angular spacing defined between some adjacent flute sections is denoted by means of the angle cc, whereas the angular spacing defined between some other adjacent flute sections is denoted by means of the angle α+δ, while the angular spacing defined between still other adjacent flute sections is denoted by means of the angle α−δ, with pairs of similar angular spacings being disposed diametrically opposite each other. The reason for such spacing is to prevent or alleviate any chatter or vibration when the compression tool 100 is being rotated. It is also noted at this juncture that the compression tool 100 of the present invention is intended to be rotated at low to moderate speeds, such as, for example, within the range of 550-900 RPM depending upon the diametrical size of the particular compression tool 100. Still further, it is seen that the plurality of flute sections 106 may, for example, comprise six (6) flute sections, although the precise number of flute sections 106 may vary.

Continuing further, and as can best be appreciated from FIG. 4, it is seen that each flute section 106 comprises a leading crest or cutting edge portion 108, a trailing margin portion 110 connected to and immediately trailing the leading crest or cutting edge portion 108, and a trailing relieved land portion 112. The trailing margin portion 110 is effectively disposed within the same tangential plane, or upon the same circumferential locus, as that of the leading crest or cutting edge portion 108, as considered in connection with the circumferential locus defined by means of the plurality of leading crest or cutting edge portions 108 or as considered in connection with the inner circumferential wall of the bone material defining the implantation site, while the trailing relieved land portion 112 is angled away from the tangentially oriented trailing margin portion 110 by means of a relief angle a so as to effectively reduce the surface area disposed in contact with the inner circumferential wall of the bone material defining the osteotomy which thereby, in turn, reduces the amount of heat generated during the cutting, compression, or densification process of the finishing procedure. It is also appreciated that the leading crest or cutting edge portion 108 also leads into an arcuately configured grooved, pocket or flute section 114 of each flute section 106 so as to effectively accumulate minute particles of bone which have been removed by means of the leading crest or cutting edge portions 108 of the flute sections 106, as opposed to being evacuated from the implantation site. This is achieved as a result of the flute sections 106 being left-handed flute sections, while the leading crest or cutting edge portions 108 are right-handed cutting edges. As a result of this integral composite structure, the leading crest or cutting edge portions 108 will therefore effectively remove minute particles of bone from the innermost circumferential wall surface of the bone surrounding the implantation site, and such particles will then be entrapped and will accordingly accumulate within the arcuately configured grooved, pocket, or flute sections 114 of the flute sections 106. The clockwise rotation of the drill or osteotome, as can be appreciated from FIGS. 3 and 4, will then effectively push or force such accumulated bone particles forwardly toward the next successive trailing margin portion 110 where such trailing margin portion 110 will then compress such minute particles of bone radially outwardly and into the bone material circumferentially surrounding the implantation site, thereby enhancing the bone density of the bone material circumferentially surrounding and defining the implantation site.

Continuing still further, it is additionally noted that the lower fluted section 116 of the shank or body portion 102 is tapered a predetermined amount as one proceeds from the distal end, at which the flute sections 106 begin, toward the opposite apical end 117 which is adapted to be inserted within a rotary motor, not shown, which will impart rotational movement to the compression tool, wherein it is to be additionally appreciated that the distal end of the compression tool 100, which terminates in a substantially flat end face 118, is provided with the smallest diametrical extent. It is further noted, that in conjunction with such tapered fluted section 116 of the compression tool 100, and the dimensions of the diametrical extents of the flute sections 106 at any axial location along the longitudinally axial extent of the compression tool 100, that the upper, non-fluted section 120 of the shank or body portion 102 has the diametrical extents, from smallest to largest, laser-marked upon one external side surface portion of such upper, non-fluted section 120 of the shank or body portion 102 of the compression tool 100, as noted at 122 and as can best be seen in FIG. 1a. In particular, it is noted that the particularly illustrated compression tool 100 has a tapered diametrical extent which continuously varies, as one proceeds in the direction extending from the distal end face 118 of the compression tool 100 toward the apical, non-fluted end 120 of the compression tool 100, from 2.5 mm to 3.5 mm. In this manner, the dental practitioner can easily select the precise compression tool 100 that he wants or needs in order to perform a particular finishing procedure in connection with a particular implantation site.

In conjunction with such laser marking 122 of the diametrical dimensions of the drill or osteotome 100 upon the non-fluted section 120 of the compression tool 100, the diametrically opposite side surface portion of the non-fluted section 120 of the compression tool 100 is also provided with an additional laser marking, as illustrated at 124 within FIG. 1b, which indicates the length of the lower fluted section 116 of the compression tool 100. In this manner, as was characteristic of the laser markings 122 for readily indicating to the dental practitioner the smallest and largest diametrical extents of the lower fluted section 116 of the drill or osteotome 100, the laser marking 124 readily indicates to the dental practitioner the axial length of the lower fluted section 116 of the compression tool 100 so that, again, the dental practitioner can likewise select the precise compression tool 100 that he desires in order to properly finish the implantation site in accordance with previously reviewed X-ray or panoramic images of the implantation site to be defined within the patient's jaw.

Still further, as can best be seen from FIGS. 1a-2, the new compression tool 100 of the present invention has a circumferentially extending flanged portion 126 formed integrally upon the shank portion 102 of the compression tool 100 at an axial location which separates the lower fluted section 116 of the compression tool 100 from the upper non-fluted section 120 of the compression tool 100. This circumferentially extending flanged portion 126 actually comprises two adjacent annularly grooved sections or bands 128,130 which are color-coded. More particularly, for example, the annular section or band 130, which is disposed closest to the distal end face 118, is colored yellow which indicates that the compression tool 100 comprises a tapered compression tool, while the annular section or band 128, which is disposed closest to the apical end 117, is colored green and indicates a particular diametrical extent range which will, of course, correspond with the laser markings 122 as defined upon one diametrical side surface portion of the non-fluted section 120 of the compression tool 100. Color coding is conventionally known to dental practitioners, however, placing such color coding indicia directly upon the axial central region of the shank portion 102 of the compression tool 100 is new and serves as a quick, convenient, visual aid to the dental practitioner when selecting the precisely proper compression tool 100 for the particular dental implant. A second purpose of the circumferentially extending flanged portion 126 is that it effectively serves as a “stopper” whereby the same effectively prevents the dental practitioner from inserting the compression tool 100 any further into the dental implantation site than is necessary whereby, alternatively further axial penetration of the compression tool 100 into the implantation site is not to be pursued or else injury to the implantation site, or to the membranes or sinus regions within the jawbone or skull, may occur.

Still yet further, reverting back to the distal flat end face 118 of the compression tool 100, and in conjunction with the circumferentially extending flanged portion 126 which serves the aforenoted “stopper” function for the compression tool 100, the distal end face 118 of the compression tool 100, being substantially flat, effectively ensures, unlike many conventional drills or osteotomes, that the fluted end 116 of the compression too1100 cannot in fact pierce any membranes or enter any sinus cavities defined within the human mandible or lower jawbone, or within the human maxilla or upper jawbone. In addition, the substantially flat distal end face 118 of the compression tool 100 provides a significant role in rebuilding lost bone mass. When human teeth deteriorate, and usually result in the loss of one or more teeth, thereby necessitating the implantation of dental implants, one adverse effect of such tooth deterioration is the loss of bone mass. Teeth may start to become loose, usually necessitating, for example, extraction. However, before a dental implant can be successfully implanted within the implantation site, which is the particular location or site from which an original tooth has been extracted, the bone mass defining the implantation site needs to be rebuilt and rendered solid such that the dental implantation will in fact be successful. As a result of the new compression tool 100, particularly characterized by means of the substantially flat distal end face 118 of the compression tool 100, bone material can be inserted into the lowermost depths of the implantation site, and then utilizing the new compression tool 100, the substantially flat distal end face 118 of the compression tool 100 can not only be used to press added bone material onto whatever bone material still exists at the implantation site so as to effectively rebuild, uplift, or enhance the presence of existing bone, but in addition, the provision of the substantially flat distal end face 118 of the compression tool 100 will in fact permit such newly added bone material to be compressed into the existing bone material and thereby render the entire bone material, surrounding the implantation site, harder and denser as a result of such compression, compaction, and densification so as together define a solid mass of bone material at the implantation site. Still further, as has been noted, this entire process can be achieved without any fear that the substantially flat distal end portion 124 of the compression tool 100 will pierce any membranes or enter any sinus cavities defined or present within the jawbones or skull of the patient.

Lastly, it is to be noted that the fluted section 116 of the compression tool 100 is coated with a DLC (diamond-like-carbon) coating. The coating is colored dark gray so as to intentionally be dull and not shiny. This permits the compression tool 100 to be utilized with conventional overhead LED dental lighting without giving off, emitting, or resulting in any light reflection which can be disturbing and annoying to the dental practitioner, as well as somewhat inhibiting to his operating procedures. The coating also effectively serves as a lubricant, thereby imparting smooth cutting during the finishing procedure due to the presence of the hard carbon lubricant content. Still yet further, the use of such coating, with its inherent lubricity, tends to reduce the temperature and heat level normally encountered during such finishing processes, thereby eliminating or significantly reducing the need for irrigation cooling.

Having described all of the structural features of the present invention compression tool 100, a finishing procedure will now be briefly described. When a dental implant procedure is to be performed, and after an original tooth has been extracted from its original position which then, of course, effectively becomes the implantation site at which a dental implant will be implanted so as to effectively replace the extracted tooth, the dental practitioner will first drill a pilot hole, within the jawbone of the patient, which is usually, for example, approximately 2.0 mm in diameter. The practitioner will then use the new compression tool 100 in order to ensure that the drilled hole or osteotomy, which will serve as the implantation site for the dental implant, has the characteristic density and hardness required to ensure that the implantation site can in fact properly support the dental implant so as to, in turn, ensure that the implantation process is successfully performed and completed whereby the dental implant will serve as a proper tooth for the patient for many years into the future. In selecting the compression tool 100, the dental practitioner will immediately note the color-coded annular sections or bands 128,130 as a result of which the dental practitioner is immediately informed that the particular compression tool 100 is a tapered compression tool and that it has diametrical extents, from the smallest to the largest, within a certain range. The particular range can be further assured as a result of the surgeon noting the laser marking 122.

At this point in time, the practitoner is ready to utilize the new compression tool 100. Due to its inherent diametrical enlargement, as a result of its taper as one proceeds from the distal end face 118 toward the apical end 117, as the practitioner inserts the compression tool 100 into the previously formed pilot hole, the bored hole will gradually increase in size as the compression tool 100 is fed axially into the bore hole. As the sharp, cutting edges 108 of each fluted section 106 encounter internal peripheral wall sections of the bore hole, bone fibers are removed from the innermost peripheral wall surface portions of the bore hole, but in lieu of such bone fibers being evacuated, such particles or fibers will effectively be entrapped and accumulated within the arcuately configured grooved, pocket, or flute sections 114 of the flute sections 106, and then the successive trailing margin portions 110 will serve to force such minute particles or fibers of the bone radially outwardly so as to compress the same into the bone material circumferentially surrounding the implantation site, thereby enhancing the bone density of the bone material circumferentially surrounding and defining the implantation site. It is to be noted that, in conjunction with the foregoing occurrences, the compression tool 100 of the present invention is only rotated in the clockwise direction as opposed to the aforenoted prior art drills or osteotomes which cut portions of the bone as a result of the drill or osteotome being rotated in the clockwise direction and then rotated in the counterclockwise direction so as to achieve compression or densification of the bone material defining the implantation site. In addition, the finishing process is completed within a single pass or as a result of the continuous insertion of the compression tool 100 into the bore hole whereby the finishing operation of the compression tool 100 is able to be achieved without any “bouncing” of the compression tool 100, or without the need for the practitioner to periodically check or gauge the depth to which he has inserted the compression tool 100 into the implantation site. This is simply achieved as a result of the practitioner selecting the proper length compression tool 100 as a result of noting or consulting the laser marking 124 disposed upon the upper, non-fluted section 120 of the shank portion 102 of the compression tool 100, as well as the provision of the “stopper” 126 upon the central section of the shank portion 102 of compression tool 100.

Obviously, many variations and modifications of the present invention are possible in light of the above teachings. It is therefore to be understood that within the scope of the appended claims, the present invention may be practiced otherwise than as specifically described herein.

1. A compression tool for performing a finishing procedure, within a bore hole formed within a human jawbone so as to serve as an implantation site for a dental implant, by compression and densification processes, comprising:
a body portion defined around a longitudinal axis, wherein a lower distal end section of said body portion is tapered such that the smallest diameter of said lower distal end section of said body portion is disposed at a distal end of said body portion;
a plurality of right-handed flute sections disposed within a circumferential array upon said lower distal end section of said body portion for continuously cutting bone material from bone mass surrounding and defining the bore hole formed within the human jawbone;
a plurality of left-handed flute sections angularly spaced from but operatively associated with said plurality of right-handed flute sections so as to define pocket sections between said plurality of right-handed flutes and said left-handed flutes for accumulating bone material cut from the bone mass surrounding and defining the bore hole formed within the human jawbone,
wherein, when said compression tool is rotated only in a clockwise direction and simultaneously axially inserted into the bore hole, said plurality of right-handed flute sections, for continuously cutting bone material from bone mass surrounding and defining the bore hole formed within the human jawbone, will continuously cut bone material from the bone mass surrounding and defining the bore hole within the human jawbone, the bone material, continuously cut from the bone mass surrounding and defining the bore hole formed within the human jawbone, will be accumulated within said pocket sections so as not to be evacuated from the implantation site, and will be immediately compressed radially outwardly back into the bone mass surrounding and defining the bore hole formed within the human jawbone so as to compress, compact, and enhance the density of the bone, mass surrounding and defining the bore hole formed within the human jawbone.

2. (canceled)

3. The compression tool as set forth in claim 1, wherein:
each one of said plurality of right-handed flute sections comprises a leading crest or cutting edge portion for cutting radially innermost bone matter from the bone mass surrounding and defining the bore hole formed within the human jawbone; and
a plurality of trailing margin portions are respectively connected to and immediately trail a respective one of said plurality of leading crest or cutting edge portions for compressing the cut bone matter back into the bone mass surrounding and defining the bore hole formed within the human jawbone so as to enhance the density of the bone mass surrounding and defining the bore hole formed within the human jawbone.

4. The compression tool as set forth in claim 3, wherein:
said plurality of leading crest or cutting edge portions for cutting radially innermost bone matter from the bone mass surrounding and defining the bore hole formed within the human jawbone are disposed within tangential planes as considered in connection with the inner circumferential wall of the bone mass surrounding and defining the bore hole formed within the human jawbone and located upon a single circular locus; and
said plurality of trailing margin portion are effectively disposed within the same tangential plane and upon the same circular locus as that of said plurality of leading crest or cutting edge portions.

5. The compression tool as set forth in claim 3, wherein each one of said plurality of flute sections further comprises:
a plurality of trailing relieved land portions respectively integrally connected to said plurality of trailing margin portions wherein said plurality of trailing relieved land portions are angled away from said tangentially oriented trailing margin portions by means of a predetermined relief angle so as to effectively reduce the surface area disposed in contact with the inner circumferential wall of the bone material defining the bore hole which thereby, in turn, reduces the amount, of heat generated during the compression or densification process of the procedure.

6. The compression tool as set forth in claim 1, wherein:
adjacent ones of said plurality of flute sections are spaced unequally from other adjacent ones of said plurality of flute sections, as considered within said circumferential array of flute sections, so as to effectively prevent vibration or chattering of said compression tool as said compression tool is rotated during said finishing procedure.

7. The compression tool as set forth in claim 1, wherein:
said compression tool comprises first indicia noted upon a a first surface portion of an upper non-fluted section of said body portion for readily visually indicating the diametrical extents, from smallest to largest, of said tapered lower distal end portion of said compression tool.

8. The compression tool as set forth in claim 1, wherein:
said compression tool comprises second indicia noted a second surface portion of said upper non-fluted section of said body portion for readily visually indicating the length dimension of said lower fluted section of said body portion.

9. The compression tool as set forth in claim 1, further comprising:
a circumferentially extending flanged portion defined upon an axially central portion of said body portion for serving as a stopper for preventing axial progression of said compression tool beyond a predetermined amount so as to prevent said compression tool from piercing any membranes or entering any sinus cavities.

10. The compression tool as set forth in claim 9, wherein:
said circumferentially extending flanged stopper comprises two circumferentially extending colored bands integrally formed thereon.

11. The compression tool as set forth in claim 10,wherein:
a first one of said two colored bands provides a visual indication that said compression tool is tapered, while a second one of said two colored bands provides a visual indication of the maximum and minimum diametrical extents of said tapered lower fluted section of said body portion.

12. The compression tool as set forth in claim 1, wherein:
an end face of said compression tool is flat whereby said compression tool cannot pierce membranes or enter sinus cavities.

13. The compression tool as set forth in claim 12, wherein:
said end face is flat so as to enable bone mass to be added to a bottom end portion of the bore hole in order to build or uplift existing bone in order to compensate for bone loss.

14. A method of performing a finishing procedure, within a bore hole formed within a human jawbone so as to serve as an implantation site for a dental implant, by compression and densification processes, comprising the steps of:
providing a compression tool with a body portion defined around a longitudinal axis, wherein a lower distal end section of said body portion is tapered such that the smallest diameter of said lower distal end section of said body portion is disposed at a distal end of said body portion, a plurality of flute sections disposed within a circumferential array upon said lower distal end section of said body portion; and
rotating said compression tool in a clockwise direction and simultaneously axially inserting said compression tool into the bore hole such that said plurality of flute sections, comprising structure for continuously cutting bone material from bone mass surrounding and defining the bore hole formed within the human jawbone, for accumulating the bone material so as to prevent the bone material from being evacuated from the implantation site, and for immediately compressing the bone material, cut from the bone mass surrounding and defining the bore hole formed within the human jawbone, back into the bone mass surrounding and defining the bore hole formed within the human jawbone, compresses, compacts, and enhances the density of the bone mass surrounding and defining the bore hole formed within the human jawbone.
